(19) United States
(12) Patent Application Publication
(10) Pub. No.: US 2022/0104899 A1
(43) Pub. Date: Apr. 7, 2022

(54) INTERFACING A SURGICAL ROBOT ARM AND INSTRUMENT

(71) Applicant: CMR SURGICAL LIMITED, Cambridge (GB)

(72) Inventors: Kenneth Focht, Needham, MA (US); Joseph Gordon, Mansfield, MA (US); Daniel P. Smith, Portsmouth, RI (US); Peter Calvin Costello, Raynham, MA (US)

(21) Appl. No.: 17/400,770

(22) Filed: Aug. 12, 2021

(30) Foreign Application Priority Data
Oct. 7, 2020 (GB) 2015914.1

(51) Int. Cl. A61B 34/00; A61B 34/30; A61B 17/28

(52) U.S. Cl. CPC A61B 34/71; A61B 2017/2939; A61B 17/28; A61B 34/30

(57) ABSTRACT

A robotic surgical instrument comprising: a shaft; driving elements running through the shaft; an articulation at a distal end of the shaft for articulating an end effector, the articulation driveable by the driving elements; and an instrument interface at a proximal end of the shaft, the instrument interface comprising instrument interface elements, each instrument interface element configured to: drive one of the driving elements, and engage a drive assembly interface element of a drive assembly interface of a surgical robot arm when the robotic surgical instrument engages the surgical robot arm such that, when engaged, both the instrument interface element and the drive assembly interface element intersect the line of the one of the driving elements.

### CROSS-REFERENCE TO RELATED APPLICATION

This application claims the benefit under 35 U.S.C. § 119 of United Kingdom Patent Application No. 2015914.1 filed on Oct. 7, 2020 which is hereby incorporated herein by reference in its entirety for all purposes.

### BACKGROUND

It is known to use robots for assisting and performing surgery. FIG. 1 illustrates a typical surgical robotic system. A surgical robot 100 consists of a base 102, an arm 104 and an instrument 106. The base supports the robot, and may itself be attached rigidly to, for example, the operating theatre floor, the operating theatre ceiling or a cart. The arm extends between the base and the instrument. The arm is articulated by means of multiple flexible joints 108 along its length, which are used to locate the surgical instrument in a desired location relative to the patient. The surgical instrument is attached to the distal end of the robot arm. The surgical instrument penetrates the body of the patient at a port so as to access the surgical site. The surgical instrument comprises a shaft connected to a distal end effector 110 by a jointed articulation. The end effector engages in a surgical procedure. In FIG. 1, the illustrated end effector is a pair of jaws.

A surgeon controls the surgical robot 100 via a remote surgeon console 112. The surgeon console comprises one or more surgeon input devices 114. These may take the form of a hand controller or foot pedal. The surgeon console also comprises a display 116.

A control system 118 connects the surgeon console 112 to the surgical robot 100. The control system receives inputs from the surgeon input device(s) 114 and converts these to control signals to move the joints of the robot arm 104 and instrument 106. The control system sends these control signals to the robot, where the corresponding joints are driven accordingly.

Some movements of the end effector 110, such as a translation, are enabled solely by articulating the robot arm 104. Other movements of the end effector 110, such as changing the pose of the end effector or opening and closing the jaws of the end effector, are enabled by articulating the joints in the articulation of the instrument. Mechanical drive for driving the instrument's joints is transferred from the robot arm to the instrument at an interface between the two.

Several instruments are typically used during the course of a surgical procedure. It is desirable for an instrument to be easily detachable from and attachable to the robot arm in order to facilitate exchange of one instrument for another on the robot arm mid-surgery. Ideally, such instrument exchange is performed quickly without the use of additional tools.

There is thus a need for a robot arm/instrument interface that provides mechanical drive from the robot arm to the instrument in a mechanically robust manner that maximises force transfer, whilst enabling the robot arm and instrument to be quickly detached and attached.

### SUMMARY OF THE INVENTION

According to an aspect of the invention, there is provided a robotic surgical instrument comprising: a shaft; driving elements running through the shaft; an articulation at a distal end of the shaft for articulating an end effector, the articulation driveable by the driving elements; and an instrument interface at a proximal end of the shaft, the instrument interface comprising instrument interface elements, each instrument interface element configured to: drive one of the driving elements, and engage a drive assembly interface element of a drive assembly interface of a surgical robot arm when the robotic surgical instrument engages the surgical robot arm such that, when engaged, both the instrument interface element and the drive assembly interface element intersect the line of the one of the driving elements.

Each instrument interface element may be shaped such that when the robotic surgical instrument engages the surgical robot arm, the instrument interface element receives the drive assembly interface element.

Each instrument interface element may have a socket shape configured to receive a plug-shaped drive assembly interface element.

The robotic surgical instrument may comprise three instrument interface elements, each instrument interface element configured to: drive a respective one of the driving elements; and engage a respective drive assembly interface element of the drive assembly interface of a surgical robot arm when the robotic surgical instrument engages the surgical robot arm such that, when engaged, both the instrument interface element and its respective drive assembly interface element intersect the line of the respective one of the driving elements.

The three instrument interface elements may be the only instrument interface elements of the instrument interface.

Each instrument interface element may be linearly displaceable along a displacement axis parallel to a longitudinal axis of the shaft.

The articulation may comprise joints for articulating the end effector, each joint driveable by one of the driving elements.

According to an aspect of the invention, there is provided a surgical robot arm comprising: a base connected to a terminal link via a series of intermediate joints, the terminal link comprising a drive assembly interface, the drive assembly interface comprising drive assembly interface elements, each drive assembly interface element configured to: engage an instrument interface element of an instrument interface of a robotic surgical instrument when the surgical robot arm engages the robotic surgical instrument such that, when engaged, both the instrument interface element and the drive assembly interface element intersect the line of a driving element driven by the instrument interface element for articulating an end effector, and drive the instrument interface element.

Each drive assembly interface element may be shaped such that when the robotic surgical instrument engages the surgical robot arm, the drive assembly interface element is received by the instrument interface element.

Each drive assembly interface element may have a plug shape configured to be received by a socket-shaped instrument interface element.

The surgical robot arm may comprise three drive assembly interface elements, each drive assembly interface element configured to: drive a respective instrument interface element; and engage a respective instrument interface element of the instrument interface of the robotic surgical instrument when the surgical robot arm engages the robotic surgical instrument such that, when engaged, both the drive assembly interface element and its respective instrument interface element intersect the line of the driving element driven by the instrument interface.

The three drive assembly interface elements may be the only drive assembly interface elements of the drive assembly interface.

Each drive assembly interface element may be linearly displaceable along a displacement axis parallel to a longitudinal axis of the terminal link.

Each drive assembly interface element may have: a distal end protruding from the drive assembly interface perpendicular to the displacement direction of the drive assembly interface element, the distal end for engagement with a respective instrument interface element; and a proximal end in the drive assembly interface, wherein the proximal end is fixedly attached to a carriage, the carriage being linearly displaceable parallel to the displacement direction of the drive assembly interface element.

The carriage may be configured to slide linearly along a track parallel to the longitudinal axis of the terminal link.

The length of the carriage parallel to the longitudinal axis of the terminal link may be greater than the length of the drive assembly interface element perpendicular to the longitudinal axis of the terminal link.

According to an aspect of the invention, there is provided a surgical robot comprising: a robotic surgical instrument comprising: a shaft; driving elements running through the shaft;

an articulation at a distal end of the shaft for articulating an end effector, the articulation driveable by the driving elements; and an instrument interface at a proximal end of the shaft, the instrument interface comprising instrument interface elements, each instrument interface element configured to drive one of the driving elements; and a surgical robot arm comprising: a base connected to a terminal link via a series of intermediate joints, the terminal link comprising a drive assembly interface, the drive assembly interface comprising drive assembly interface elements, each drive assembly interface element configured to: engage an instrument interface element when the surgical robot arm engages the robotic surgical instrument such that, when engaged, both the instrument interface element and the drive assembly interface element intersect the line of the driving element driven by the instrument interface element, and drive the instrument interface element.

Each drive assembly interface element may be linearly displaceable along a displacement axis parallel to the longitudinal axis of the terminal link.

The shaft may have a longitudinal axis, and the terminal link may have a longitudinal axis, wherein the longitudinal axis of the shaft is parallel to the longitudinal axis of the terminal link.

The longitudinal axis of the shaft may be colinear with the longitudinal axis of the terminal link.

### BRIEF DESCRIPTION OF THE FIGURES

The present invention will now be described by way of example with reference to the accompanying drawings. In the drawings:

FIG. 1 illustrates a surgical robot system for performing a surgical procedure;

FIG. 2 illustrates a surgical robot;

FIG. 3 illustrates an exemplary surgical instrument;

FIGS. 4a and 4b illustrate the distal end of an exemplary surgical instrument;

FIGS. 5a and 5b illustrate the instrument interface at the proximal end of an exemplary surgical instrument;

FIGS. 6a and 6b illustrate the drive assembly interface at the distal end of an exemplary surgical robot arm; and

FIGS. 7a and 7b illustrate the instrument interface of FIGS. 5a and 5b engaged with the drive assembly interface of FIGS. 6a and 6b.

### DETAILED DESCRIPTION

The following describes an interface between a surgical robotic arm and a surgical instrument.

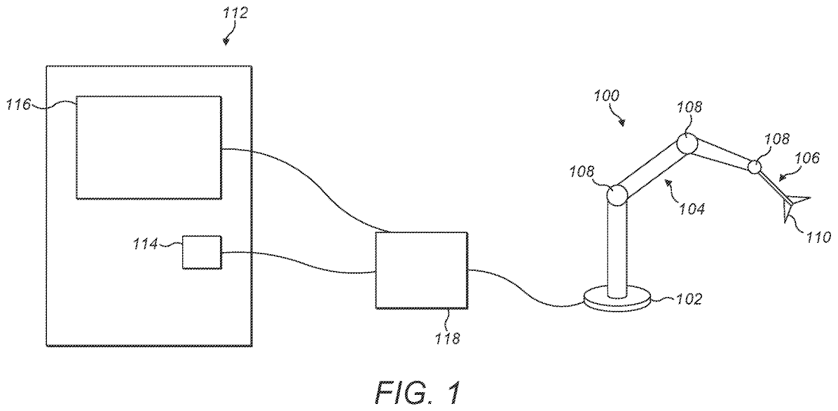

The surgical robotic arm and surgical instrument form part of a surgical robotic system of the type illustrated in FIG. 1.

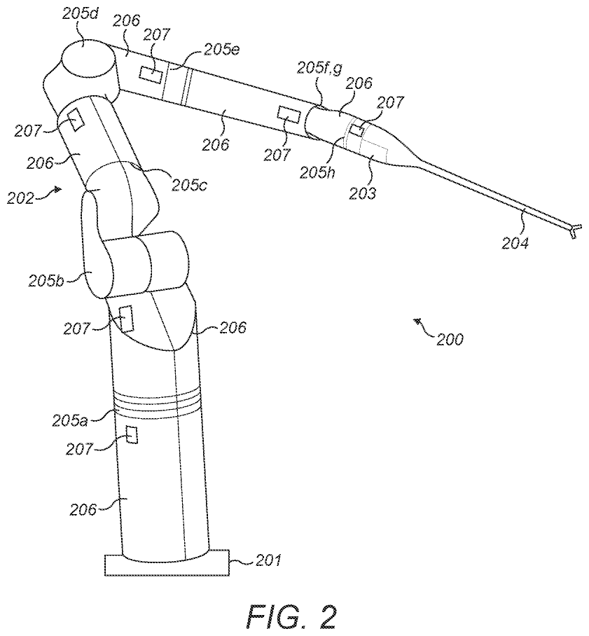

FIG. 2 illustrates an example robot 200. The robot comprises a base 201 which is fixed in place when a surgical procedure is being performed. Suitably, the base 201 is mounted to a chassis. That chassis may be a cart, for example a bedside cart for mounting the robot at bed height. Alternatively, the chassis may be a ceiling mounted device, or a bed mounted device.

A robot arm 202 extends from the base 201 of the robot to a terminal link 203 to which a surgical instrument 204 can be attached. The arm is flexible. It is articulated by means of multiple flexible joints 205 along its length. In between the joints are rigid arm links 206. The arm in FIG. 2 has eight joints. The joints include one or more roll joints (which have an axis of rotation along the longitudinal direction of the arm members on either side of the joint), one or more pitch joints (which have an axis of rotation transverse to the longitudinal direction of the preceding arm member), and one or more yaw joints (which also have an axis of rotation transverse to the longitudinal direction of the preceding arm member and also transverse to the rotation axis of a co-located pitch joint). In the example of FIG. 2: joints 205a, 205c, 205e and 205h are roll joints; joints 205b, 205d and 205f are pitch joints; and joint 205g is a yaw joint. Pitch joint 205f and yaw joint 205g have intersecting axes of rotation. The order of the joints from the base 201 to the terminal link 203 of the robot arm is thus: roll, pitch, roll, pitch, roll, pitch, yaw, roll. However, the arm could be jointed differently. For example, the arm may have fewer than eight or more than eight joints. The arm may include joints that permit motion other than rotation between respective sides of the joint, for example a telescopic joint. The robot comprises a set of drivers 207. Each driver 207 has a motor which drives one or more of the joints 205. The terminal link 203 of the robot arm comprises a drive assembly for interfacing and driving a surgical instrument. The drive assembly will be described in more detail below.

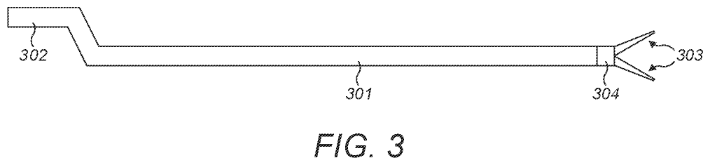

FIG. 3 illustrates a surgical instrument 204. The surgical instrument has an elongate profile, with a shaft 301 spanning between its proximal end which is attached to the robot arm and its distal end which accesses the surgical site within the patient body. Suitably, the shaft is rigid. The shaft may be straight. The proximal end of the surgical instrument and the instrument shaft may be rigid with respect to each other and rigid with respect to the distal end of the robot arm when attached to it. At the proximal end of the instrument, the shaft 301 is connected to an instrument interface 302. The instrument interface engages with the drive assembly interface at the distal end of the robot arm as will be described in more detail below. At the distal end of the surgical instrument, the distal end of the shaft is connected to an end effector 303 by an articulation 304. The end effector 303 engages in a surgical procedure at the surgical site. The end effector may take any suitable form. For example, the end effector could be a pair of curved scissors, an electrosurgical instrument such as a pair of monopolar scissors, a needle holder, a pair of jaws, or a fenestrated grasper.

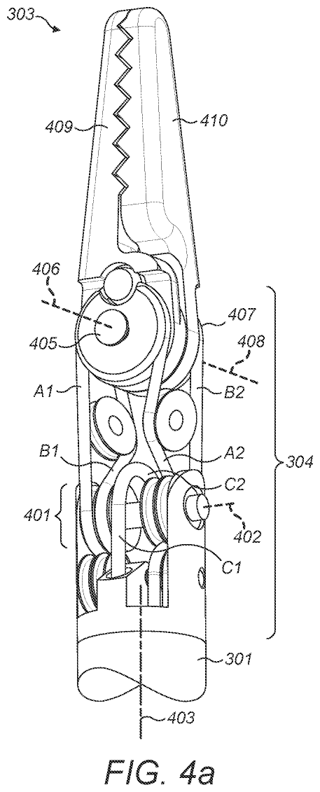

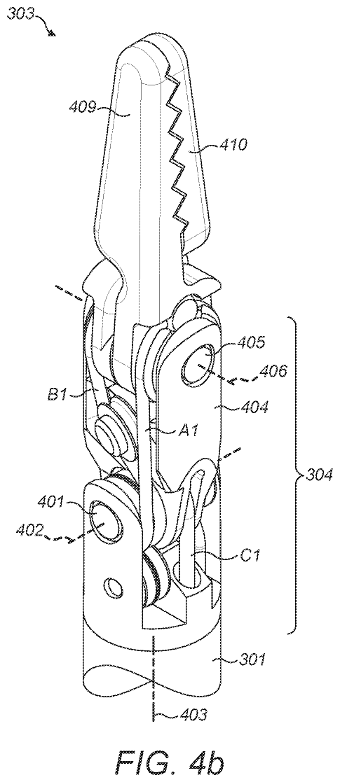

FIGS. 4a and 4b illustrate the distal end of an exemplary instrument which has a pair of jaws as the end effector 303. The shaft 301 is connected to the end effector 303 by articulation 304. The articulation 304 comprises several joints. These joints enable the pose of the end effector to be altered relative to the direction of the instrument shaft. Although not shown in FIGS. 4a and 4b, the end effector may also comprise joint(s). In the example of FIGS. 4a and 4b, the articulation 304 comprises a pitch joint 401. The pitch joint 401 rotates about pitch axis 402, which is perpendicular to the longitudinal axis 403 of the shaft 301. The pitch joint 401 permits a supporting body 404 (described below) and hence the end effector 303 to rotate about the pitch axis 402 relative to the shaft. In the example of FIGS. 4a and 4b, the articulation also comprises a first yaw joint 405 and a second yaw joint 407. First yaw joint 405 rotates about first yaw axis 406. Second yaw joint 407 rotates about second yaw axis 408. Both yaw axes 406 and 408 are perpendicular to pitch axis 402. Yaw axes 406 and 408 may be parallel. Yaw axes 406 and 408 may be collinear. The articulation 304 comprises a supporting body 404. At one end, the supporting body 404 is connected to the shaft 301 by pitch joint 401. At its other end, the supporting body 404 is connected to the end effector 303 by the yaw joints 405 and 407. This supporting body is omitted from FIG. 4a for ease of illustration so as to enable the other structure of the articulation to be more easily seen.

The end effector 303 shown comprises two end effector elements 409, 410. Alternatively, the end effector may have a single end effector element. The end effector elements 409, 410 shown in FIGS. 4a and 4b are opposing jaws. However, the end effector elements may be any type of opposing end effector elements. The first yaw joint 405 is fast with the first end effector element 409 and permits the first end effector element 409 to rotate about the first yaw axis 406 relative to the supporting body 404 and the pitch joint 401. The second yaw joint 407 is fast with the second end effector element 410 and permits the second end effector element 410 to rotate about the second yaw axis 408 relative to the supporting body 404 and the pitch joint 401. In FIGS. 4a and 4b, the end effector elements 409, 410 are shown in a closed configuration in which the jaws abut.

The joints illustrated in FIGS. 4a and 4b are driven by pairs of driving elements. The driving elements are elongate. They are flexible transverse to their longitudinal extent. They resist compression and tension forces along their longitudinal extent. A first pair of driving elements A1, A2 are constrained to move around the first yaw joint 405. Driving elements Al, A2 drive rotation of the first end effector element 409 about the first yaw axis 406. FIGS. 4a and 4b illustrate a second pair of driving elements B1, B2 which are constrained to move around the second yaw joint 407. Driving elements B1, B2 drive rotation of the second end effector element 410 about the second yaw axis 408. FIGS. 4a and 4b also illustrate a third pair of driving elements C1, C2 which are constrained to move around pitch joint 401. Driving elements C1, C2 drive rotation of the end effector 303 about the pitch axis 402. The end effector 303 can be rotated about the pitch axis 402 by applying tension to driving elements C1 and/or C2. The pitch joint 401 and yaw joints 405, 407 are independently driven by their respective driving elements.

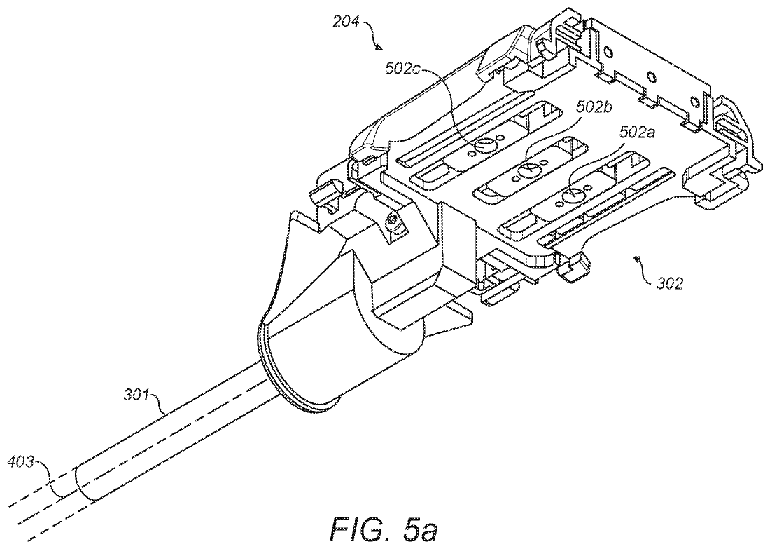

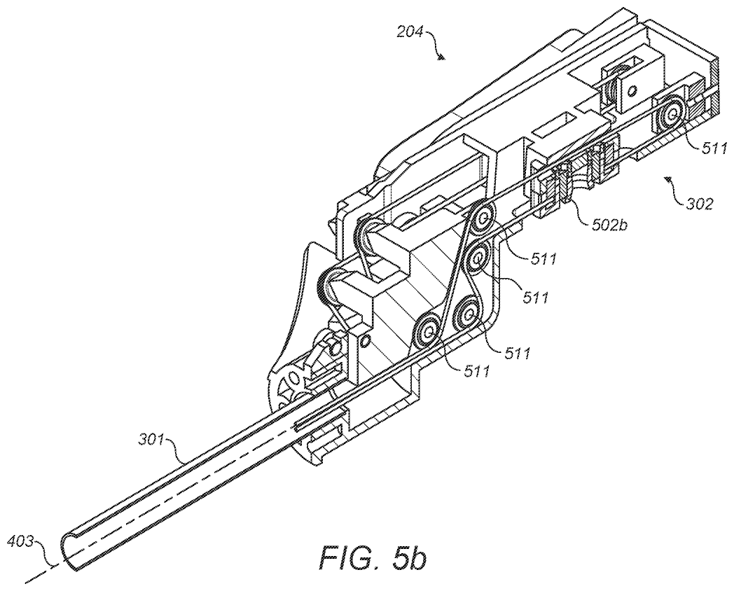

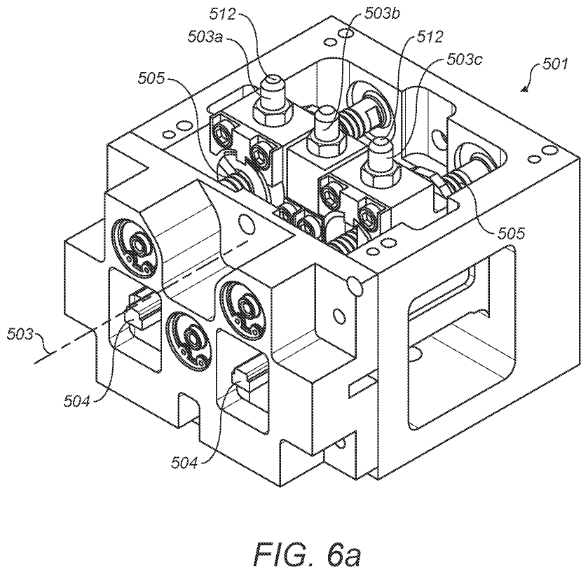

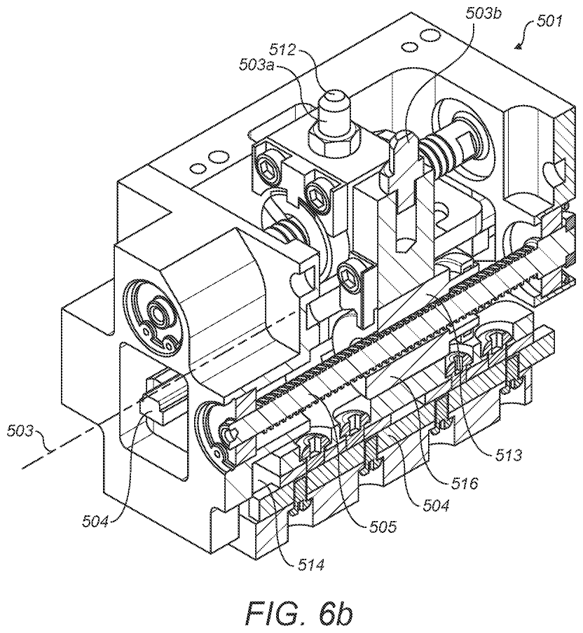

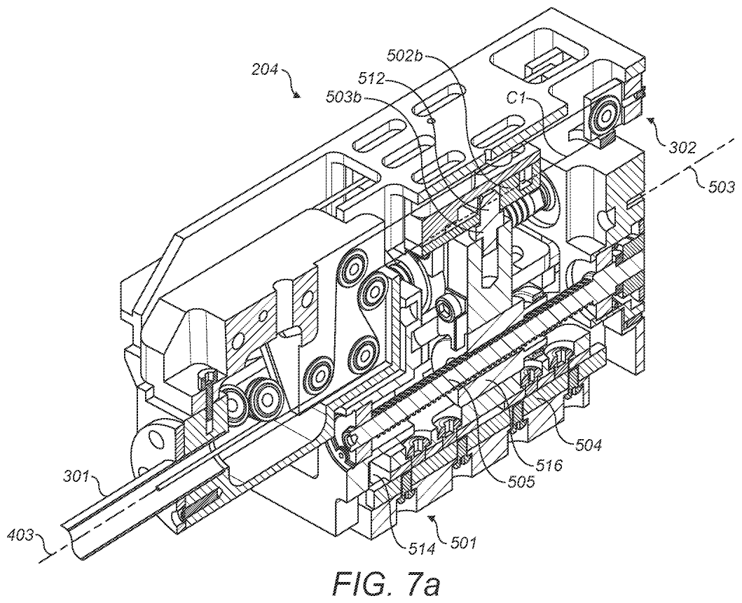

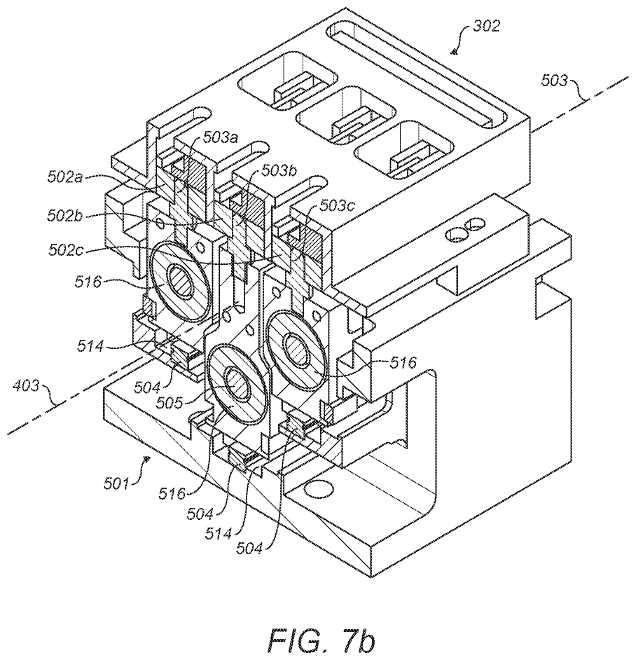

FIGS. 5a and 5b illustrate the instrument interface 302 at the proximal end of the surgical instrument. FIGS. 6a and 6b illustrate the drive assembly interface 501 at the distal end of the surgical robot arm 200. FIGS. 7a and 7b illustrate the instrument interface 302 and drive assembly interface 501 when engaged. In FIGS. 7a and 7b, the surgical instrument 204 is attached to the robot arm 200, and the instrument interface 302 is engaged with the drive assembly interface 501. FIG. 5b illustrates a cross section through the instrument interface in a plane parallel to the longitudinal axis 403 of the shaft 301 of the instrument. FIG. 6b illustrates a cross section through the drive assembly interface in a plane parallel to the longitudinal axis 503 of the terminal link of the robot arm. FIG. 7a illustrates a cross section through the engaged interfaces of the robot arm and instrument in a plane parallel to the longitudinal axis 503 of the terminal link 203 of the robot arm. The plane of the cross section includes the longitudinal axis 503 of the terminal link 203, and is perpendicular to the plane in which the instrument interface and drive assembly interface engage. FIG. 7b illustrates a cross section through the engaged interfaces in a plane perpendicular to the longitudinal axis 503 of the terminal link 203 of the robot arm. This cross section is also in a plane perpendicular to the longitudinal axis 403 of the shaft 301 of the instrument.

The instrument interface 302 comprises instrument interface elements 502a, 502b, 502c. Each instrument interface element 502a, 502b, 502c is secured to a respective driving element or pair of driving elements. In other words, each instrument interface element is fast with a respective driving element or pair of driving elements such that displacement of the instrument interface element is transferred to displacement of the driving element/pair of driving elements. In the example that the distal end of the instrument is as shown in FIGS. 4a and 4b, instrument interface element 502a may be secured to pair of driving elements A1, A2, instrument interface element 502b may be secured to pair of driving elements C1, C2 and instrument interface elements 502c may be secured to pair of driving elements B1, B2. Each instrument interface element is linearly displaceable along a displacement axis. In the figures shown, this displacement axis is parallel to the longitudinal axis 403 of the instrument shaft 301. Thus, in response to being driven itself, each instrument interface element linearly drives the driving element or pair of driving elements to which it is secured parallel to the longitudinal axis of the instrument shaft. Alternatively, each instrument interface element may be displaceable along a displacement axis which is at an angle to the longitudinal axis of the instrument shaft. Further structure in the instrument interface, such as a pulley mechanism, may be used to bring the driving elements into the proximal end of the shaft such that the driving elements pass through the shaft parallel to the longitudinal axis of the shaft. FIGS. 5a, 5b, 7a and 7b illustrate three instrument interface elements. These three instrument interface elements may be the only instrument interface elements which are used to transfer mechanical drive to the instrument joints. Alternatively, there may be more than three or fewer than three instrument interface elements which are used to transfer mechanical drive to the instrument joints.

The driving elements pass down the instrument shaft 301, and exit the instrument shaft into the instrument interface 302. If the instrument interface element to which that driving element is secured is not in line with the driving element as it exits the shaft, then the driving element is routed through the instrument interface so as to meet the instrument interface element. For example, as shown in FIG. 5b, the driving element may be constrained to move about a set of pulleys 511 so as to bring it into contact with the instrument interface element.

The driving elements may be cables. The driving elements may comprise flexible portions and a rigid portion. Flexible portions engage the components of the instrument interface and the instrument articulation, and the rigid portion extends through all or part of the instrument shaft. For example, the flexible portion may be a cable, and the rigid portion may be a spoke. Other rigid portion(s) may be in the instrument interface or instrument articulation. For example, rack and pinions may be in the instrument interface or instrument articulation.

The drive assembly interface 501 comprises drive assembly interface elements 503a, 503b, 503c. Each drive assembly interface element 503a, 503b, 503c is linearly displaceable along a displacement axis. In the figures shown, this displacement axis is parallel to the longitudinal axis 503 of the terminal link 203 of the robot arm. Each drive assembly interface element is driven linearly along its displacement axis by a motor in the drive assembly. For example, as will be described in more detail below, each drive assembly interface element may be driven along a linear track 504 by a lead screw 505, the lead screw being driven to rotate by the motor. Each drive assembly interface element may be driven by a different motor to the other drive assembly interface elements. Alternatively, one or more drive assembly interface element may be driven by the same motor. FIGS. 6a and 7b illustrates three drive assembly interface elements. These three drive assembly interface elements may be the only drive assembly interface elements which are used to transfer mechanical drive to the instrument. Alternatively, there may be more than three or fewer than three drive assembly interface elements which are used to transfer mechanical drive to the instrument.

The instrument interface and drive assembly interface are shaped relative to one another such that, when they are engaged, each drive assembly interface element engages a respective instrument interface element. In the example shown in FIGS. 5, 6 and 7, instrument interface element 502a engages drive assembly interface element 503a, instrument interface element 502b engages drive assembly interface element 503b, and instrument interface element 502c engages drive assembly interface element 503c.

In the example shown in FIGS. 5, 6 and 7, the drive assembly interface elements 503a, 503b, 503c protrude from the remaining profile of the drive assembly, and the instrument interface elements 502a, 502b, 502c are recessed into the remaining profile of the instrument interface. The shape and location of each drive assembly interface element is complimentary to the shape and location of a corresponding instrument interface element, such that when the instrument interface and drive assembly interface are engaged, the drive assembly interface element is received in the instrument interface element. FIGS. 5a, 5b, 7a and 7b illustrate the instrument interface element having a socket shape, and the distal end of the drive assembly interface element having a plug shape. The socket-shaped instrument interface element is configured to receive the plug-shaped drive assembly interface element. This may be a plug-fit or snug-fit.

In an alternative implementation (not shown), the instrument interface elements protrude from the remaining profile of the instrument interface, and the drive assembly interface elements are recessed into the remaining profile of the drive assembly interface. The shape and location of each drive assembly interface element is complimentary to the shape and location of a corresponding instrument interface element, such that when the instrument interface and drive assembly interface are engaged, the instrument interface element is received in the drive assembly interface element. For example, the drive assembly interface element may have a socket shape, and the distal end of the instrument interface element may have a plug shape. In this example, the socket-shaped drive assembly interface element is configured to receive the plug-shaped instrument interface element. This may be a plug-fit or snug-fit.

Each instrument interface element may directly contact its corresponding drive assembly interface element when the surgical instrument engages the robot arm. In this case, the instrument interface element and corresponding drive assembly interface element may be complimentarily shaped such that when engaged, they form a plug-fit or snug-fit. Such a tight fit reduces slipping between the drive assembly interface element and instrument interface element, thereby increasing the efficiency of the transfer of drive from the drive assembly interface element to the instrument interface element.

Alternatively, a sterile barrier may be located between the instrument interface element and its corresponding drive assembly interface element. This sterile barrier may, for example, form part of a drape used to cover the robot arm. A patient undergoing surgery is only exposed to a sterile environment. The instruments are either newly used for each patient or sterilised between uses. However, the robot arm is treated as non-sterile because it cannot be cleaned between uses to a sterile degree. Thus, the robot arm is covered in a sterile barrier, typically a drape. To maintain a fully sterile environment, this sterile barrier extends between the instrument interface and the drive assembly interface, and hence between each instrument interface element and the drive assembly interface element that it engages.

Thus, the instrument interface element may contact one face of the sterile barrier, and the drive assembly interface element contact the opposing face of the sterile barrier. The combination of the drive assembly interface element, sterile barrier, and instrument interface element is, as a whole, a plug-fit or snug-fit. For example, the plug-shaped distal end of the drive assembly interface element may be received in the socket-shaped face of the sterile barrier as a plug-fit; and the opposing plug-shaped face of the sterile barrier may be received in the socket-shaped face of the instrument interface element as a plug-fit.

When the instrument is attached to the robot arm, and the instrument interface is engaged with the drive assembly interface as described above, the longitudinal axis of the instrument shaft 403 is parallel to the longitudinal axis of the terminal link 503. As shown in FIGS. 7a and 7b, the longitudinal axis of the instrument shaft 403 may be colinear with the longitudinal axis of the terminal link 503. Thus, as each drive assembly interface element is driven linearly along its displacement axis by a motor in the drive assembly, by virtue of its engagement with its corresponding instrument interface element, it linearly drives the instrument interface element parallel to the longitudinal axis of the terminal link. Thus, the robot arm transfers mechanical drive to the end effector as follows: movement of a drive assembly interface element moves an instrument interface element which moves a driving element which moves one or more joints of the articulation and/or end effector which moves the end effector.

When each drive assembly interface element is engaged with its respective instrument interface element, both the drive assembly interface element and the instrument interface element intersect the line of the driving element secured to the instrument interface element. This is illustrated on FIG. 7a, where both the engaged instrument interface element 502b and drive assembly interface element 503b intersect the line 509 of driving element C1 secured to instrument interface element 502b. In other words, both instrument interface element 502b and drive assembly interface element 503b intersect the longitudinal axis of the driving element C1 in the vicinity of where it is secured to the instrument interface element 502b.

Each drive assembly interface element and its corresponding instrument interface element intersecting along the line of the driving element secured to the instrument interface element when engaged results in a more efficient transfer of drive from the motor of the drive assembly to the driving element. The drive is transferred in the intended direction, i.e. down the line of the driving element. Further components in the instrument interface to cause the instrument interface element to move in the intended linear direction, such as a guide rail, are not necessary since the drive assembly interface element performs this function. Thus, the frictional losses associated with the contact between the instrument interface element and such further components are not incurred. Hence the transfer of drive is more efficient.

The drive assembly interface elements shown in FIGS. 6a, 6b, 7a and 7b each have a distal end 512 which protrudes from the drive assembly interface perpendicular to the longitudinal axis of the terminal link. It is the distal end 512 of the drive assembly interface element which engages with its respective instrument interface element.

The drive assembly interface element has a proximal end 513 which is located in the drive assembly interface. The proximal end 513 comprises a cylinder 516 through which the lead screw 505 passes. The lead screw 505 is in threaded engagement with the threaded interior surface of the cylinder 516. The proximal end 513 is fixedly attached to a carriage 514. The carriage is linearly displaceable parallel to the longitudinal axis 503 of the terminal link of the robot arm. In the example shown, the carriage 514 is constrained to move along a track 504. The track 504 runs parallel to the longitudinal axis 503 of the terminal link. The carriage is constrained to move along the linear track by any suitable means. For example, the carriage may comprise rollers for rolling along the track. Alternatively, or in addition, the track may comprise rollers for facilitating movement of the carriage along the track. The carriage may take the form of an outer cylindrical structure which slides over a track which is in the form of a cylindrical rail. The drive assembly interface element is thus prevented from rotating as the lead screw rotates by virtue of it being fixed to the carriage 514 which cannot rotate but only move linearly along the track 504.

The result is that as the lead screw rotates, the rotation is transferred to linear motion of the carriage along the track, and hence the linear motion of the drive assembly interface element in the same direction as the carriage 514. Rotation of the lead screw in one rotational direction causes the drive assembly interface element, and hence the instrument interface element it is engaged with to displace linearly towards the end effector. Rotation of the lead screw in the opposing rotational direction causes the drive assembly interface element, and hence the instrument interface element it is engaged with to displace linearly away from the end effector.

The carriage 514 has a length in the direction parallel to the longitudinal axis of the terminal link which is greater than the length of the drive assembly interface element in the direction perpendicular to the longitudinal axis of the terminal link and perpendicular to the plane in which the instrument interface and drive assembly interface engage. For example, the carriage may have a length between 3.5 and 4 cm, whereas the length of the instrument interface element between its proximal and distal ends may be between 2 and 3.5 cm. The length of the proximal end of the drive assembly interface element in the direction parallel to the longitudinal axis of the terminal link may be wider than the length of the distal end of the drive assembly interface element in the direction parallel to the longitudinal axis of the terminal link. For example, the proximal end may be between 2 and 2.5 cm in length, whereas the distal end may be between 0.2 and 1 cm in length.

Bending moments in the distal end of the drive assembly interface element are induced by driving and resistive forces applied by the sterile barrier and/or instrument interface element 502. These cause moments between the lead screw and the drive assembly interface element. Thus, some energy can be lost in an undesirable rocking motion of the drive assembly interface element. By making the proximal end of the drive assembly interface element wider than the distal end, and the carriage wider than the distal end, rocking of the distal end of the drive assembly interface element is reduced. This improves the efficiency of the drive transfer from the robot arm to the instrument. Ideally, the length of the carriage 514 in the direction of the longitudinal axis of the terminal link is maximised, thereby minimising the undesirable rocking motion.

The proximal end of the drive assembly interface element 513 may be symmetrically fixed to the carriage 514 along the length of the carriage 514 in the direction parallel to the longitudinal axis 503 of the terminal link. Alternatively, the proximal end of the drive assembly interface element 513 may be fixed to the carriage 514 closer to one end of the carriage than the other. For example, the proximal end of the drive assembly interface element 513 may be fixed to the carriage 514 closer to the end of the carriage that is closest to the motor (i.e. furthest from where the instrument attaches). The middle of the drive assembly interface element may be located between 30% and 45% along the length of the carriage from the motor end of the carriage. For example, the middle of the drive assembly interface element may be located 40% of the way along the length of the carriage from the motor end of the carriage.

Resistive forces may be applied asymmetrically to the distal end of the drive assembly interface element. For example, if linear motion of the distal end of the drive assembly interface element, and hence the engaged instrument interface element, in a first direction causes a closing motion of the end effector, this may incur more resistance than linear motion of the distal end of the drive assembly interface element in a second, opposing direction which causes an opening motion of the end effector. Thus, motion of the drive assembly interface element in the first direction corresponds to a greater bending moment of the drive assembly interface element than motion of the drive assembly interface element in the second direction. As a result of this known asymmetry in the exerted bending moments, the drive assembly interface element may be asymmetrically fixed to the carriage 514 along the length of the carriage. Thus, a greater resistance to the rocking of the distal end of the drive assembly interface element is provided for the direction subject to the greater magnitude of rocking.

The moments that the carriage 514 supports are zero because the carriage 514 is free to move along the track 504. If the proximal end 513 of the drive assembly interface element is fixed to the carriage 514 asymmetrically along the length of the carriage 514, then less force perpendicular to the direction of the longitudinal axis 503 of the terminal link would be exerted on the end of the carriage which protrudes further from the proximal end of the drive assembly interface element than the other end of the carriage. Thus, the end of the carriage which protrudes further from the proximal end of the drive assembly interface element may be located in a portion of the drive assembly which is weaker than the portion of the drive assembly housing the other end of the carriage.

Between the proximal end 513 and the distal end 512 of the drive assembly interface element, the body of the drive assembly interface element is linear and straight. Suitably, the drive assembly interface element is very stiff. For example the Young's modulus of the drive assembly interface element may be between 100 and 300 GPa. The Young's modulus of the drive assembly interface element may be 200 GPa. The drive assembly interface element may be fabricated from corrosion treated stainless steel, such as corrosion treated 17-4PH stainless steel.

By making the drive assembly interface element very stiff, bending moments experienced by the distal end 512 cause insignificant bending of the drive assembly interface element. Instead the moments are transferred to the carriage 514 where they are resisted as forces perpendicular to the longitudinal axis of the carriage, where their impact on drive inefficiencies is reduced.

The instrument interface is releasably engageable with the drive assembly. The instrument can be detached from the robot arm manually without requiring any tools. This enables the instrument to be detached from the drive assembly quickly and another instrument attached during an operation. The instrument interface and drive assembly interface may attach to each other via the engagement of the instrument interface elements and drive assembly interface elements. This may be in addition to other complimentary shaped surface features on the instrument interface and drive assembly interface, such as protrusions on the drive assembly interface and corresponding recesses on the instrument interface. Alternatively, a separate engagement mechanism may be used to lock the instrument interface to the drive assembly interface.

Thus, the interface between the surgical robot arm and the surgical instrument described herein provides mechanical drive from the robot arm to the instrument in a mechanically robust manner that maximises force transfer, whilst enabling the robot arm and instrument to be quickly detached and attached.

The robot described herein could be for purposes other than surgery. For example, the port could be an inspection port in a manufactured article such as a car engine and the robot could control a viewing tool for viewing inside the engine.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims. The applicant indicates that aspects of the present invention may consist of any such individual feature or combination of features. In view of the foregoing description it will be evident to a person skilled in the art that various modifications may be made within the scope of the invention.

What is claimed is:

1. A robotic surgical instrument comprising:
a shaft;
driving elements running through the shaft;
an articulation at a distal end of the shaft for articulating an end effector, the articulation driveable by the driving elements; and
an instrument interface at a proximal end of the shaft, the instrument interface comprising instrument interface elements, each instrument interface element configured to:
drive one of the driving elements, and
engage a drive assembly interface element of a drive assembly interface of a surgical robot arm when the robotic surgical instrument engages the surgical robot arm such that, when engaged, both the instrument interface element and the drive assembly interface element intersect the line of the one of the driving elements.

2. A robotic surgical instrument as claimed in claim 1, wherein each instrument interface element is shaped such that when the robotic surgical instrument engages the surgical robot arm, the instrument interface element receives the drive assembly interface element.

3. A robotic surgical instrument as claimed in claim 2, wherein each instrument interface element has a socket shape configured to receive a plug-shaped drive assembly interface element.

4. A robotic surgical instrument as claimed in claim 1, comprising three instrument interface elements, each instrument interface element configured to:
drive a respective one of the driving elements; and
engage a respective drive assembly interface element of the drive assembly interface of a surgical robot arm when the robotic surgical instrument engages the surgical robot arm such that, when engaged, both the instrument interface element and its respective drive assembly interface element intersect the line of the respective one of the driving elements.

5. A robotic surgical instrument as claimed in claim 4, wherein the three instrument interface elements are the only instrument interface elements of the instrument interface.

6. A robotic surgical instrument as claimed in claim 1, wherein each instrument interface element is linearly displaceable along a displacement axis parallel to a longitudinal axis of the shaft.

7. A robotic surgical instrument as claimed in claim 1, wherein the articulation comprises joints for articulating the end effector, each joint driveable by one of the driving elements.

8. A surgical robot arm comprising:
a base connected to a terminal link via a series of intermediate joints, the terminal link comprising a drive assembly interface, the drive assembly interface comprising drive assembly interface elements, each drive assembly interface element configured to:
engage an instrument interface element of an instrument interface of a robotic surgical instrument when the surgical robot arm engages the robotic surgical instrument such that, when engaged, both the instrument interface element and the drive assembly interface element intersect the line of a driving element driven by the instrument interface element for articulating an end effector, and
drive the instrument interface element.

9. A surgical robot arm as claimed in claim 8, wherein each drive assembly interface element is shaped such that when the robotic surgical instrument engages the surgical robot arm, the drive assembly interface element is received by the instrument interface element.

10. A surgical robot arm as claimed in claim 9, wherein each drive assembly interface element has a plug shape configured to be received by a socket-shaped instrument interface element.

11. A surgical robot arm as claimed in claim 8, comprising three drive assembly interface elements, each drive assembly interface element configured to:
drive a respective instrument interface element; and
engage a respective instrument interface element of the instrument interface of the robotic surgical instrument when the surgical robot arm engages the robotic surgical instrument such that, when engaged, both the drive assembly interface element and its respective instrument interface element intersect the line of the driving element driven by the instrument interface.

12. A surgical robot arm as claimed in claim 11, wherein the three drive assembly interface elements are the only drive assembly interface elements of the drive assembly interface.

13. A surgical robot arm as claimed in claim 8, wherein each drive assembly interface element is linearly displaceable along a displacement axis parallel to a longitudinal axis of the terminal link.

14. A surgical robot arm as claimed in claim 8, wherein each drive assembly interface element has:
a distal end protruding from the drive assembly interface perpendicular to the displacement direction of the drive assembly interface element, the distal end for engagement with a respective instrument interface element; and
a proximal end in the drive assembly interface, wherein the proximal end is fixedly attached to a carriage, the carriage being linearly displaceable parallel to the displacement direction of the drive assembly interface element.

15. A surgical robot arm as claimed in claim 14, wherein the carriage is configured to slide linearly along a track parallel to the longitudinal axis of the terminal link.

16. A surgical robot arm as claimed in claim 14, wherein the length of the carriage parallel to the longitudinal axis of the terminal link is greater than the length of the drive assembly interface element perpendicular to the longitudinal axis of the terminal link.

17. A surgical robot comprising:
a robotic surgical instrument comprising:
a shaft;
driving elements running through the shaft;
an articulation at a distal end of the shaft for articulating an end effector, the articulation driveable by the driving elements; and
an instrument interface at a proximal end of the shaft, the instrument interface comprising instrument interface elements, each instrument interface element configured to drive one of the driving elements; and
a surgical robot arm comprising:
a base connected to a terminal link via a series of intermediate joints, the terminal link comprising a drive assembly interface, the drive assembly interface comprising drive assembly interface elements, each drive assembly interface element configured to:
engage an instrument interface element when the surgical robot arm engages the robotic surgical instrument such that, when engaged, both the instrument interface element and the drive assembly interface element intersect the line of the driving element driven by the instrument interface element, and
drive the instrument interface element.

18. A surgical robot arm as claimed in claim 17, wherein each drive assembly interface element is linearly displaceable along a displacement axis parallel to the longitudinal axis of the terminal link.

19. A surgical robot as claimed in claim 17, the shaft having a longitudinal axis, and the terminal link having a longitudinal axis, wherein the longitudinal axis of the shaft is parallel to the longitudinal axis of the terminal link.

20. A surgical robot as claimed in claim 19, wherein the longitudinal axis of the shaft is colinear with the longitudinal axis of the terminal link.
